(19) United States
(12) Patent Application Publication
(10) Pub. No.: US 2026/0141978 A1
(43) Pub. Date: May 21, 2026

(54) BIOINFORMATICS METHOD FOR DETERMINING THERAPEUTIC TARGET REGIONS

(71) Applicants: Centre National de la Recherche Scientifique, PARIS (FR); Université Paris Cité, PARIS 6 (FR); ECOLE NATIONALE SUPERIEURE DE CHIMIE DE PARIS, PARIS CEDEX 05 (FR)

(72) Inventors: Anne VANET, Paris (FR); Michel PETITJEAN, Paris (FR); Aurélie PERRIER PINEAU, Juvisy-sur-Orge (FR); Valentin OZEEL, Alfortville (FR); Julie LAO, Noisy-le-Grand (FR)

(21) Appl. No.: 19/113,692

(22) PCT Filed: Sep. 19, 2023
(86) PCT No.: PCT/FR2023/051429

§ 371 Date: Mar. 20, 2025

(30) Foreign Application Priority Data
Sep. 20, 2022 (FR) FR2209473

(51) Int. Cl. G16B 15/30; G16B 15/20; G16B 40/20

(52) U.S. Cl. CPC G16B 15/30; G16B 15/20; G16B 40/20

(57) ABSTRACT

A computer-implemented method includes identifying, in a set of previously aligned nucleotide and polypeptide sequences characteristic of a candidate protein, which can be referred to as target residues; identifying at least one candidate region consisting of at least one pair of target residues identified in the first step that include target residues located at a determined distance in space and being exposed at the surface of the candidate protein; determining the advantageous chemical interactions between each residue and/or between each pair of residues within the candidate protein, from the 2D and/or 3D structure of the candidate protein that are hydrophobic bonds and/or hydrogen bonds and/or saline bridges and/or negative-repulsion and/or positive-repulsion bonds; selecting the residues linked by said advantageous chemical interactions that are at a distance of at most 10 angstroms; and selecting at least one therapeutic target region from among the candidate regions that include the selected residues.

### CROSS-REFERENCE TO RELATED APPLICATIONS

The present application is a filing under 35 U.S.C. 371 as the National Stage of International Application No. PCT/FR2023/051429, filed Sep. 19, 2023, entitled “BIOINFORMATICS METHOD FOR DETERMINING THERAPEUTIC TARGET REGIONS,” which claims priority to French Application No. 2209473 filed with the Intellectual Property Office of France on Sep. 20, 2022, both of which are incorporated herein by reference in their entirety for all purposes.

### TECHNICAL FIELD

The present application relates to a bioinformatics method for identifying reliable and durable therapeutic target regions, in order to optimize the search for new drugs, notably antivirals.

### STATE OF THE ART

Despite the existence of treatments, RNA viruses still represent a serious public health problem. Indeed, their high mutation rate enables them to rapidly acquire resistance to these treatments. To prevent the emergence of resistance, it is recommended to target invariant amino acids as a priority. Indeed, mutations in highly conserved positions lead to deterioration or alteration of biological functions and could render the virus non-viable. However, due to their very small number, invariant positions alone cannot constitute binding sites for a drug.

To find other optimal binding sites accessible to a drug, Lao J. et al. propose to also identify pairs of covariant mutations called “synthetic lethals” (SL) [Brouillet et al., Petitjean et al.]. SLs represent mutations which are not lethal but which, when combined, render the virus non-viable. These SLs have already been studied in the search for anti-cancer drugs [Kuiken H. J., and Beijersbergen R. L] and anti-HIV agents. Lao et al. propose a series of computational steps in order to identify the best target residues which, when mutated or blocked by a drug, could substantially affect the biological function of the targeted pathogen.

However, the method proposed by Lao et al. has its drawbacks. Firstly, the targets identified by this method are not described with sufficient precision, which penalizes the user in his development program. For example, this method does not reveal whether the target proposed by the software has a large number of invariant residues, a specific volume or is unlikely to mutate in the future. Furthermore, it does not take into account the fact that the batch of initial sequences may not be very usable or, on the contrary, may be very reliable and therefore highly predictive. Finally, this method lacks a crucial piece of information for establishing the relevance of the targets identified: the nature of the chemical bonds that may exist between the residues of the candidate protein and more particularly in the target region. This biochemical information would make it possible to reinforce the validity of the targets identified by the genetic approach of Lao et al. and to select the most relevant.

Typically, the number of therapeutic target regions identified by virtue of genetic screening by Lao et al. is too high. Since research organizations then have to set up long and costly screening programs for libraries of molecules (antibodies, small molecules, etc.), it is preferable to reduce the number of target regions of interest as much as possible, selecting only those that are likely to be long-lasting and stable, notably by taking into account existing biochemical links within the protein of interest.

### DESCRIPTION OF THE INVENTION

According to a first aspect, the invention aims to improve the method of Lao et al. To achieve this, the present inventors propose to integrate into this method steps based on the 3D structure and the intramolecular chemical interactions of the target protein. These steps make it possible to confirm or validate the residues identified by the Lao et al. method, depending on the physico-chemical quality of their interactions and their sensitivity to environmental parameters (pH, temperature, etc.). By virtue of these steps, only those pairs of residues having advantageous physico-chemical characteristics and located at such a distance that the chemical links between residues potentially have an influence, will be selected.

In addition, the present inventors propose to apply filters to the Lao et al. method in order to exclude nucleotide or peptide sequences of insufficient quality from the analysis, and thus avoid burdening the system by working on unusable or poorly indexed sequences. The present inventors have thus developed additional steps for more precise selection of the sequences to be tested and/or to be used first.

Finally, a method of this type must be able to generate results quickly, whether on proteins from different microorganisms, or on proteins from different variants of the same microorganism. It is therefore important to be able to have a fast and safe method for processing several proteins in parallel, enabling notably the 3D structures of proteins from known variants to be taken into account.

All of these developments make it possible to improve the reliability and the interest of the method described in Lao et al., which was theoretical but difficult to use (as it was insufficiently described and poorly documented). The method of the present invention is more effective and more reliable than that described in Lao et al., in that it has been scientifically enriched and supplemented by new criteria enabling the targets detected to be described in detail, both statistically and biochemically. By virtue of these improvements, the method of the invention becomes essential for identifying important target regions in pathogenic organisms, with a view to enabling researchers to design tomorrow's drugs.

For this purpose, a computer-implemented method is proposed for determining at least one therapeutic target region on a candidate protein of a target pathogenic organism, said method comprising the following steps:
a) Identifying, in a set of previously aligned nucleotide and polypeptide sequences characteristic of said candidate protein, invariant residues and/or pairs of synthetic lethal residues referred to as target residues;
b) Identifying at least one candidate region consisting of at least one pair of target residues identified in step a), said at least one pair comprising target residues located at a determined distance in space and being exposed at the surface of the candidate protein, preferably in a pocket;
c) Determining the advantageous chemical interactions between each residue and/or between each pair of residues within the candidate protein, from the 2D and/or 3D structure of the candidate protein; said advantageous chemical interactions being hydrophobic bonds and/or hydrogen bonds and/or salt bridges and/or negative-repulsion and/or positive-repulsion bonds;
d) Selecting the residues linked by said advantageous chemical interactions, said residues being at a distance of at most 10 angstroms;
e) Selecting at least one therapeutic target region from among the candidate regions identified in step b), comprising the residues selected in step d).

The invention, according to the first aspect, is advantageously completed by the following features, taken alone or in any of their technically possible combinations:
the step of identifying advantageous chemical interactions consists in determining the chemical interactivity of each residue within the protein and/or determining the chemical interactivity of each pair of residues separated by a determined distance;
the distance determined in step b) is between 2 and 8 angstroms, preferably 5 angstroms;
the method comprises an evaluation of the chemical interactivity of the determined bonds comprising a calculation of at least one score from: a percentage of hydrophobic bonds and/or a percentage of hydrogen bonds and/or, a percentage of salt bridges and/or a percentage of negative repulsion bonds and/or a percentage of positive repulsion bonds and/or a percentage of chemical bonds;
the target region is a pocket identified from a set of aligned 3D structures of the candidate protein and wherein chemical interactions are determined on a set of aligned 3D structures of the candidate protein;
the target pocket comprises at least five target residues selected from synthetic lethal invariant residues or covariant residues, and wherein said pocket has a volume between 60 \dot{A}³ to 500 \dot{A}³;
the method comprises an evaluation of the quality of the spatial location of the target residues, said evaluation being characterized by the calculation of the proportion of pairs of residues located within 5 angstroms of each other and/or the proportion of pairs of residues located within 10 angstroms of each other;
the method comprises a step of selecting the reference polypeptide sequence of the candidate protein, followed by a step of selecting the polypeptide sequences to be tested having lengths identical to the reference sequence, and/or having a number of mutations less than or equal to three standard deviations of the mean number of mutations per sequence, and/or having an invariant residue at its N or C terminal;
the method comprises an evaluation of the quality of the initial sequences selected, said evaluation comprising the calculation of at least one score measuring the heterogeneity of the length of the initial sequences and/or the number of sequences having an aberrant number of poorly-defined amino acids and/or the number of sequences having an aberrant number of missing residues.

According to a second aspect, the invention aims to identify a therapeutic target region based solely on the chemical interactions that exist between residues in the region. For this purpose, a computer-implemented method is proposed for determining at least one therapeutic target region on a candidate protein of a target pathogenic organism, said method comprising the following steps:
a) Determining the advantageous chemical interactions between each residue and/or between each pair of residues within the candidate protein, from the 2D and/or 3D structure of the candidate protein; said advantageous chemical interactions are hydrophobic bonds and/or hydrogen bonds and/or salt bridges and/or negative-repulsion and/or positive-repulsion bonds;
b) Selecting the residues linked by said advantageous chemical interactions, said residues being at a distance of at most 10 angstroms, said residues forming a therapeutic target region.

### DESCRIPTION OF THE FIGURES

Further features, aims and advantages of the invention will become apparent from the following description, which is purely illustrative and non-limiting, and should be read in conjunction with

FIG. 1, which shows a diagram of the steps of a computer-implemented method for determining at least one therapeutic target region on the surface of a candidate protein of a pathogenic organism.

FIG. 2 shows a diagram of the chemical interactions existing between pairs of amino acids within the HA protein of the influenza virus (HB=hydrogen bond, SB=salt bridge, PR=positive repulsion, HI=hydrophobic interaction). The highlighted bonds are those corresponding to pairs already defined by the technique described in Lao et al and therefore confirmed by the chemical method of the invention, linking pairs or groups of amino acids whose distance is suitable (highlighted in gray); these pairs will therefore be selected as part of the method of the invention.

FIGS. 3A-3F highlight the amino acids of interest selected in FIG. 2 within the amino acids identified after implementing steps E1 and E2 of the method of the invention (for further details, see FIGS. 3A-3F of Lao et al.)

### DETAILED DESCRIPTION OF THE INVENTION

The present invention relates to a computer-implemented method for determining at least one therapeutic target region on the surface of a candidate protein of a pathogenic organism. Such a method can for example be implemented by a processing unit such as one or more processors or any other equivalent means.

The term “target pathogenic organism” is used herein to refer to any type of organism capable of causing disease in a “host” (such as a human, a plant or an animal). This organism is preferably a microorganism such as a virus, a bacterium, a parasite, a fungus, a protozoan (amoeba, sporozoan or flagellate), etc. Some of these organisms have only been sequenced in recent decades, such as:
rotavirus, Calicivirus (Norwalk and Hepatitis E), Ebola, Chikungunya, SARS Coronavirus;
legionella, Campylobacter, Helicobacter, Mycobacterium and Escherichia coli strain O157: H7;
sporozoa (parasitic protozoa) of the orders Coccidia (Cryptosporidium, Cyclospora, Toxoplasma) or Microsporidia (Enterocytozoon, Encephalitozoon, Nosema).

It may also be a harmful macroorganism, such as a worm (belonging for example to the group of helminths, platyhelminthes such as trematodes or cestodes, or nemathelminthes such as the nematodes Ascaris, Toxocara, or Trichuris) or an insect.

By extension, the term “target pathogenic organism” also includes tumor cells or cells infected by a pathogenic microorganism such as a virus, a bacterium, etc. Indeed, these cells often express on their surface or in their cytoplasm (or even in their nuclei) proteins involved in maintaining proliferation signals for these cells, resisting cell death, escaping the immune system, angiogenesis, activating invasion and metastasis, replicative immortality, escaping growth factor suppressors, reprogramming energy metabolism, thereby amplifying the disease (cancer or infection). It therefore makes perfect sense to use the method of the invention to identify target regions suitable for therapeutic research, on proteins that are involved in these disorders too.

Pathogenic organisms are essentially made up of proteins, some of which are necessary for their development, infectivity and/or pathogenicity. For every known pathogen, numerous proteins of this type have been identified and constitute a prime target for researchers in the pharmaceutical industry. Indeed, disabling the function and/or masking such proteins often makes it possible to halt the development, propagation and/or deleterious effects of pathogens on human, plant or animal health. In the context of the present application, these proteins will be referred to as “candidate proteins”, in that they have been previously identified as candidates having a potential impact on the development, infectivity and/or pathogenicity of a pathogenic organism of interest.

For a molecule to be selected as a “drug”, it must have a substantial influence, in the short, medium or long term, on the function of at least one of these candidate proteins. To do this, it must first be able to make contact with the candidate protein (if possible, by virtue of several contact zones). By virtue of 3D structures, it is now possible to determine which zones are localized on the protein surface, and therefore which zones may be possible “contact zones” between a drug molecule and a protein of interest. For each protein, however, these contact zones are too numerous to be screened in all research programs. To facilitate their work and speed up the identification of effective drugs, researchers need to know more precisely which “target regions” on the surface of candidate proteins have the most promising chemical and biological properties to be targeted by a drug. In the present application, a “therapeutic target region” is therefore defined as a zone of preferred contact between a drug and a candidate protein, said zone having been selected whereby the interaction between these two elements (the drug on the one hand and the protein on the other) can be chemically strong, stable and significantly influence the biological function of the protein.

### BRIEF DESCRIPTION OF THE STEPS OF THE METHOD OF THE INVENTION

It is assumed beforehand that a candidate protein known to influence the development, infectivity and/or pathogenicity of the pathogenic organism under study has already been identified. To implement the method of the invention, this candidate protein must be known and well described in the literature. Notably, polypeptide sequences and 3D sequences must have already been characterized in the art, and be readily available. The nucleotide sequences encoding these polypeptide sequences must also be known. All these sequences are generally provided in official and openly accessible databases. The sequences in these databases, hereinafter referred to as “BDD1”, are generally anonymized, freely accessible and uploaded by various international research units. Examples include Genbank, NCBI, INSDC, EMBL, HIVDB, LANL, FLUDB, GISAID, etc., all of which are well known to the skilled person.

In a step prior to the method of the invention, a polypeptide sequence and a reference nucleotide sequence must be selected (step E0). These reference sequences can be selected from a phylogenetic tree (in this case, the reference sequence is at the root of the tree representing the sequences studied) or, if the tree does not exist, by recalculating an ancestral sequence, reconstructed by one of the three following methods: parsimony, maximum likelihood or Bayesian method. This reference sequence can further be a consensus sequence from the batch of sequences studied, but in this case it only serves to be compared with the other sequences, since a calculated consensus sequence can be a sequence that has never existed, so it is not necessarily functional.

Subsequently, the other nucleotide and polypeptide sequences known/recorded for this candidate protein are identified in the “BDD1” databases. These additional sequences will hereinafter be referred to as “initial sequences”.

A protein has to perform a certain number of functions which can only be achieved if it adopts a certain structure in space and has the right chemical radicals in the right place. This is called the structure-function link. Thus certain mutations change the structure of the protein and thus cause it to lose one or more functions. If these functions are essential, the virus becomes non-replicating and can therefore no longer develop. These mutations are mainly of three kinds: invariant positions, pairs of synthetic lethals and pairs of compensatory mutations. A single mutated invariant position renders the protein non-functional, whereas to achieve the same goal, two positions must be mutated in the case of pairs of synthetic lethals. Finally, a pair of compensatory mutations is defined as follows: a first mutation renders the protein non-functional but a second could appear and restore the function of the protein studied. In these three cases, a high level of stress is imposed on the protein.

In the present invention, these initial sequences are first processed to select invariant residues and/or pairs of synthetic lethal residues (step E1). Once these invariant residues and/or pairs of synthetic lethal residues are known, at least one candidate region is identified, based on other structural criteria (step E2). Steps E1 and E2 were described by Lao et al.

In the context of the present application, a “candidate region from E2” is a region containing at least two or three amino acids of interest which have been selected to be invariant residues and/or synthetic lethal (SL) covariant residues not derived from a common ancestor, close to each other, exposed on the surface of the candidate protein and possibly in a pocket, by virtue of steps E1 and E2 of the present application, i.e. according to the method described in Lao et al.

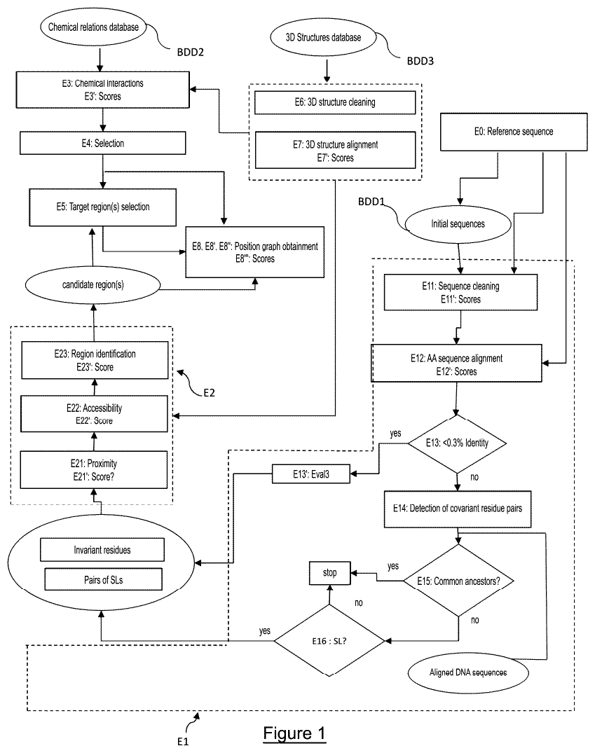

In a second step, the method of the invention provides for determining the chemical bonds involved overall between the residues of the protein, and notably between the residues located in the candidate region (steps E3, E4, E5 in FIG. 1). By virtue of this chemical interactivity information, at least one candidate region most likely to be an effective therapeutic target is identified. The method of the invention thus makes it possible to select, from the candidate regions obtained by following the indications of Lao et al., the therapeutic target region(s) most likely to enable the identification of effective drugs.

As will be detailed below, one or more scores can be advantageously calculated to check that the information obtained from each step of the method of the invention is relevant to the rest of the method and with respect to the expected results. These scores provide researchers with information on the quality and reliability of the results obtained. For the sake of readability of the description, detailed score expressions are given at the end of the description.

### Detailed Description of the Steps of the Invention

Step E1 consists in identifying, in a set of nucleotide and polypeptide sequences, characteristic of said candidate protein, and previously cleaned and aligned, the invariant residues and/or the pairs of synthetic lethal covariant residues, which will be called, in the context of the invention, “target residues”.

Databases used to store the sequences of pathogenic organisms may contain erroneous sequences which, if not eliminated, could generate false results.

This is why, in the method of the invention, the initial sequences must first be “cleaned” (step E11). This “cleaning” consists of filtering the initial sequences, for example as follows:
Initial sequences having a length different from the sequence selected as reference can be excluded (if the batch of initial sequences contains sufficient sequences);
If the batch of initial sequences does not contain many sequences and if alignment seems possible (because the sequences do not contain any hypervariable regions, and few poorly described regions), an alignment can be carried out to homogenize the lengths. In practice, from sequences of varying lengths, an alignment is obtained of a single length, which is greater than the length of the sequence having the greatest length.
Initial sequences having a number of mutations greater than three standard deviations from the mean number of mutations per sequence are excluded.

In addition, the N-terminal and C-terminal ends of the initial sequences are marked and the sequences are oriented in the same direction, so that their ends can be superimposed (for example all sequences can be oriented from the N-terminal end to the C-terminal end).

This cleaning step E11 also makes it possible to identify which of the set of initial sequences have heterogeneous lengths and/or enables sequences with unacceptable anomalies to be eliminated. Such anomalies are, for example, an aberrant number of mutations, an aberrant number of poorly-defined amino acids, an aberrant number of missing amino acids, etc. In this respect, one or more scores or score functions can be advantageously calculated (step E11′) to assess whether the initial sequences (before cleaning) do not contain too many anomalies and/or whether their length is sufficiently homogeneous (scores S1, S2, S3, S4 detailed below). If one or more of these scores is not acceptable (value close to 0 and not to 1), the method of the invention can be interrupted, as this means that the set of initial sequences before cleaning is not sufficiently robust to be exploitable.

A score function denoted f(S1QS) can also be calculated at this stage, in order to assess whether the set of initial sequences before cleaning is sufficiently complete and robust to effectively predict the existence of a therapeutic target region within the selected candidate protein. This score function f(S1QS) reflects the impact of sequence data on the quality of the result obtained at the end of the method.

It is also recommended to stop the method when the number of mutations belonging to the batches of initial sequences retained after “cleaning” is too low (typically, a number of mutations that does not allow statistically correct results to be obtained, for example that does not allow the calculation of χ² (covariants having fewer than 5 representatives)). In this case, it is preferable to select a new set of sequences from the BDD1 database, enrich it by downloading new sequences, or change the candidate protein. Conversely, a good set of initial sequences is considered to exist when at least 1000 sequences of acceptable quality have been identified, these sequences carrying sufficient mutations.

“Acceptable quality” herein means initial sequences having a number of anomalies less than three standard deviations from the mean number of these anomalies per sequence. In this respect, the score S7 can be calculated to measure the total number of initial sequences that do not contain an aberrant number of mutations. Such a score S7 depends on scores S2, S3 and S4 presented hereinbefore.

The sequences obtained after cleaning are then advantageously aligned (step E12). Sequence alignment can be implemented in two ways:
i) either the sequences are all of the same length: in this case, the alignment is generated by aligning all the sequences at their N-terminal end,
ii) or they do not all have the same length: in this case, it is difficult to use multiple alignment methods, which are generally used for a smaller number of sequences. In this case, it is possible to select a representative sample of the population of sequences studied and to use the HMMER suite (http://hmmer.org) to generate a profile whereupon all the sequences can then be aligned one by one (using the hmmbuild and hmmcalibrate functions). This method enables hundreds of thousands of sequences to be aligned in the space of a minute.

The quality of the sequence alignment can be evaluated (step E12′) by calculating one or more scores (detailed below) relating to the impact of gaps in the alignment (score S8), the redundancy of the sequence batch (score S10), the impact of hypervariable regions (score S11), the impact of deletions and insertions (score S12), the impact of post-translational modifications (score S13), and/or the impact of the existence of different subtypes (score S14). Scores S11, S12 and S13 are defined on the basis of data available in the literature.

One or more score functions can be calculated at this stage to evaluate whether the set of sequences aligned after cleaning is sufficiently complete and robust to effectively predict the existence of a therapeutic target region within the selected candidate protein. The score function f(S3) detailed below reflects the quality of the alignment and of the possible prediction. Some terms of this function describe the precision with which the initial sequences are described and have an impact on the statistical results (f(S3QS) and other terms of this function show the heterogeneity of the batch of sequences studied and have an impact on the description of the target itself f(S3SC). More precisely, the score function f(S3QS) reflects the impact of the alignment on the statistical prediction of the target region, and the score function f(S3SC) reflects the impact of the alignment on the prediction of the target.

If the predicted quality of the alignment is low, the method can be interrupted in order to be restarted from new initial sequences.

At the end of this alignment step, the user is presented with a set of cleaned and aligned sequences known as “test sequences”.

In these test sequences, invariant residues are then identified (step E13). By definition, “invariant residues” are amino acids that do not change (or hardly change) position within the protein, in all of the test sequences studied. Since sequencing methods are not 100% reliable, an average error rate of 0.3% can be applied (Cheng C. et al, 2022). Thus, in the context of the present invention, a residue is defined as “invariant” if it is present at the same position on at least 99.7% of the test sequences.

The quality of the selection of invariant residues can advantageously be assessed by calculating the mutational richness of the sequences (step E13′). Notably, score S18 can be calculated (see below). By virtue of this score, it is possible to check that the sequences on which invariant residues have been detected are sufficiently heterogeneous. Indeed, to be able to assert that a residue is invariant for functional reasons (mutations have appeared at this position, but have not been selected because they were lethal), it is necessary to be able to show that mutations have appeared elsewhere in the sequence.

Once the number of invariant residues is known, it is advantageous to determine the percentage of these residues by calculating score S19. This score is used to assess the impact of invariant positions on the final result. The targets most likely to be stable in the long term (therefore unable to mutate without modifying the replicative activity of the virus, therefore preventing the appearance of mutations that could render mutants resistant to treatment) are those that are the most invariant, therefore made up of the greatest number of invariant residues.

It is also possible to calculate the score function ƒ(S_{invariance}) which evaluates the degree of invariance of the batch of sequences studied, and gives an idea of the long-term mutational incapacity thereof. This degree of invariance depends on several variables such as: the quality of the alignment, the total number of mutations, the number of invariants, the number of synthetic lethals (which represent an invariance with two residues) and the number of mutations that occurred for functional reasons and not due to the presence of a common ancestor.

As specified in FIG. 1, other residues of interest can also be selected in the method of the invention. These are residues which are not invariant, but which belong to pairs of covariants of interest (step E14).

Several statistical tests can be used to define the covariation of a pair of variables in a list of variables. The χ² statistical test is one thereof. It is used to determine, according to a threshold, whether variables taken in pairs are independent of each other. In the present invention, it is notably possible to use the χ² by Noivirt defined as χ²(Ai, Bj) (where A and B are the specific amino acids found at positions i and j), which takes into account each of the 20 residues and not just the mutated or non-mutated state of the original residue. Once this χ²(Ai, Bj) has been defined, it is preferable to readjust the results to reject false positives due to the multiplicity of tests performed. Thus, the p-values can be readjusted using a method known as the “false discovery rate”. The residues are considered to be “dependent” or “covariant” if their p-value is below 0.05 (Noivirt O. et al.).

The impact of the number of covariant positions as well as the impact of the number of pairs of covariant positions on the final result can be evaluated by calculating scores S20 and S21 respectively.

The pairs of covariant residues identified by this calculation must then be filtered (steps E15 and E16).

As a first step, it is preferable to eliminate the pairs of covariant residues that share common ancestors (step E16). This can notably be achieved by studying the DNA sequences encoding the initial sequences: after aligning these DNA sequences, mutated codons causing non-synonymous mutations are identified and selected. In fact, non-synonymous mutations result in the appearance of amino acids that are physicochemically different, whereas synonymous mutations code for the same residue. A so-called Lewontin coefficient of linkage disequilibrium D′ can then be calculated with the set of recoded DNA sequence data (differentiating synonymous and non-synonymous positions). Using this coefficient, pairs sharing the same ancestor, whose covariation is therefore not the consequence of functional interdependencies, can be identified. By virtue of these steps, it is possible to determine whether the covariation of the two residues identified as “covariants” in step E14 results from the coevolution of these two residues or whether it is due to the fact that they are phylogenetically linked to a common ancestor. In the latter case, the residue pairs are not conserved in the method of the invention, resulting in a “false positive” known as “ancestral linkage disequilibrium” (Lao et al.; Petitjean et al.).

At this stage, it is possible to calculate scores S24 and S25 which accurately reflect the impact of overall non-synonymous mutability and the impact of synonymous mutability.

Finally, it is advantageous to identify whether any of the covariant pairs selected at the end of step E14 are capable of inducing the virus to be unable to replicate (step E16). These particular covariant pairs are known as “synthetic lethals”. In fact, it is important to distinguish between the two types of covariant mutations that exist, and which have completely opposite consequences: compensatory mutations (CM) and synthetic lethals (SL) (Lao et al.; Petitjean et al.). To do this, it is possible for example to calculate a so-called dissimilarity coefficient ξ, which allows the χ²(Ai, Bj) test to be assigned a sign. This sign differentiates between CM and SL. CMs have a ξ which is positive when NobsA,i,B,j≥NexA,i,B,j with A and B two residues located at positions i and j respectively. Thus ξA,i,B,j=+χ²(Ai, Bj) while SLs have a which is negative when NobsA,i,B,j≥NexA,i,B,j. Thus ξA,i,B,j=−χ²(Ai, Bj), with Nobs the number of pairs of residues A and B observed at positions i and j, Nex the number of pairs of residues A and B expected at positions i and j (Petitjean et al.).

At this stage, it is possible to calculate score S22, which reflects the impact of the number of SLs and their strength on the result obtained at the end of the method. Similarly, score S23 can also be calculated, as it reflects the impact of the number of CMs and their strength on the result. The strength of a pair of covariants is defined by its χ²(Ai, Bj). Indeed, the higher the χ²(Ai, Bj), the greater the number of pairs observed with respect to the number of pairs expected if there were no covariation. S22 and S23 therefore define the strength of the covariation for this particular pair.

At the end of these various steps, invariant residues and pairs of synthetic lethals are selected as part of at least one “candidate region” of the candidate protein studied.

It is herein possible to calculate score S9 which reflects the impact of variance at the 5′ and 3′ ends of the sequences. Indeed, in the step which consists of aligning DNA sequences to identify covariant residues that share a common ancestor, one of the two techniques used consists of aligning all sequences at their 5′ end. However, the variance at this end reduces the chance of aligning the sequences correctly. In this case, it may be preferable to align on the 3′ end, which in turn should be of low variation.

The candidate region selected as the best therapeutic target must further satisfy a certain number of other advantageous conditions. Notably, the amino acids of which it is composed may be stressed by the 3D structure of the candidate protein. Conversely, it may be advantageous to determine whether the candidate region contains a “pocket” that would allow a drug to lodge in a stable and strong manner. To evaluate these aspects, the method of the invention includes a step E2 which evaluates the quality of the candidate regions obtained in step E1 with regard to the position of the amino acids making them up, with respect to the 3D structure of the candidate protein.

Step E2 therefore consists in identifying, from the regions and residues identified in step E1, at least one pair of target residues located at a close distance in space, and being exposed on the surface of the candidate protein, preferably in a pocket.

To be able to bind a small molecule, a therapeutic target must be composed of residues that are spatially close to each other. Also, in the context of the present invention, the target residues of the candidate region will preferably be at most 10 angstroms apart, preferably at most 5 angstroms.

It is herein advantageous to quantify the target residues that are less than 5 angstroms and/or 10 angstroms apart by calculating different scores, in order to assess whether they are sufficient in number to constitute a future target capable of binding a potential small molecule (drug). Scores S28, S29, S30, S31 can notably be calculated (step E21′).

To obtain this information, the method of the invention advantageously requires access to known and recorded three-dimensional structures of the candidate protein. These structures are known and described in dedicated 3D structure databases, referred to herein as “BDD2”. As in the case of nucleotide and polypeptide sequences, these 3D sequences will often have to be processed prior to the method of the invention (cleaning step E6, alignment step E7).

Indeed, 3D structures are often in pdb format. However, this format is not applied in the same way by the entire scientific community (format of the file itself, names of subunits or numbering of residues, etc.). Thus, structures are preferably cleaned (step E6) to define a single and generalized format for all pdb files used (standardization of file format, numbering of residues and atoms, subunit names and their respective positions inter alia).

Furthermore, dozens or even hundreds of structures of the same protein are available. In the same way as for sequences, 3D structures recorded for the same protein are preferably aligned. These are structural alignments that reveal just how different the structures recorded for this protein are. Alignment is based on a reference model. The mean deviation between all these structures is then calculated, which is called “RMSD”.

A three-dimensional structure is defined by the position in space of each of its constituent atoms. If many of these positions are missing (missing data), the 3D structure is flawed in the sense that only some of these atoms have a fixed place in the structure. If a lot of data is missing for each of the structures studied (bearing in mind that the missing data are not at the same position in the space of the protein), it becomes difficult to make a structural alignment and even to compare residues at the same position. The fact that a protein has several subunits further complicates its structure. Moreover, in this case, we're talking about a quaternary structure and not just a tertiary structure. In this case, not only must the position of each atom in each subunit be defined, but also the position of each subunit in relation to each other.

Under these conditions, the quality of the 3D structures can therefore be evaluated to ensure that they are effective in predicting the existence of a target region. Several scores are thus calculated (step E7′). In this respect, it is possible to calculate score S5 which quantifies the number of 3D structures having an aberrant number of missing data and score S6 which evaluates the impact of the existence (if any) of different subunits on the final result.

Scores S15, S16 and S17 are also used to evaluate the alignment of 3D structures. For structures, S15 is the equivalent of Sseq for sequences. We look to see whether the number of known 3D structures for the candidate protein is high or not. If there are fewer than 200 known 3D structures, this number is insufficient for a consistent average (this figure can be reduced, however, as 3D structures become increasingly reliable).

S16 gives an idea of the variation in structure resolutions. In fact, 3D structures are determined using a variety of techniques. The two most widely used are X-ray diffraction and electron microscopy. A resolution threshold is defined for each of these structures, depending on the technique used. S16 evaluates this variation in resolution. S17 shows the structural heterogeneity of the batch of structures studied. Each of the atoms in the structure is aligned with its corresponding atom in the next structure. Thus for each atom, it is possible to associate a number of positions equal to the number of structures studied.

A mean value for this position in space is calculated, along with the deviation from the mean. If this calculation is carried out for all the atoms in the structure, it becomes possible to calculate a mean deviation that gives an idea of the heterogeneity of the structures, spatially speaking. The TM described in this score is a variant of the RMSD which normalizes it so that it is not dependent on the total number of positions in the structure.

It is also possible to calculate score functions to evaluate the quality of the structural alignment via functions ƒ(S₄), ƒ(S_{4QS}), ƒ(S_{4Qc}). Function ƒ(S₄) reflects the quality of the alignment and the possible prediction. Some terms in this function describe the precision with which the 3D structures are described and have an impact on the statistical results ƒ(S_{4QS}) and other terms of this function show the heterogeneity of the batch of sequences studied and have an impact on the description of the target itself ƒ(S_{4QC}). More precisely, the score function ƒ(S_{4QS}) reflects the impact of the alignment on the statistical prediction of the target region and the score function ƒ(S_{4QC}) reflects the impact of alignment on the prediction of the target.

From the 3D coordinates of all the atoms of residues in space, pairs of residues that are close in space, i.e. less than 5 or 10 angstroms apart, are selected (step E21).

In addition, the accessibility of target residues and/or their exposure on the surface of proteins must be taken into account. An effective therapeutic target must not be buried in the 3D structure of the protein, otherwise the drug will not be able to reach it. Thus, based on the three-dimensional structure of the candidate protein, residues that are embedded in the protein are distinguished from those that are exposed on its surface and therefore accessible (to do this, the ASA program can be used, for example). In the present method, only candidate regions containing at least two, and preferably at least three, accessible residues are selected (step E22).

Herein again, accessibility scores can be calculated to evaluate the possibility of obtaining sufficient candidate region(s) (step E22′). Score S32 evaluates the percentage of accessible positions and score S33 evaluates the percentage of accessible residues.

Finally, it may be advantageous to evaluate whether the previously selected candidate region has a 3D structure akin to a “pocket” (step E23). To do this, it is possible for example to use structure prediction software such as Fpocket (Le Guilloux et al.), wherein, in order to take account of the existence of small and large pockets, it is preferable to reduce the minimum and maximum radii of the alpha spheres to 2.5 Å and 4 Å respectively.

The Fpocket software (Le Guilloux et al., 2009) can be used to determine all pockets, whose cardinal is p̅o̅c̅k̅e̅t̅ on the surface of a protein (by providing a three-dimensional structure as the input).

The volume of a pocket able to house a small drug molecule preferably meets the following constraints: 60 Å³<pocket<500 Å³ (p̅o̅c̅k̅e̅t̅₆̅₀̅₋̅₅̅₀̅₀̅).

It is possible to calculate the percentage of pockets meeting this criterion for the protein studied S_{pocket}=p̅o̅c̅k̅e̅t̅₆̅₀̅₋̅₅̅₀̅₀̅÷p̅o̅c̅k̅e̅t̅. It is also possible to determine the sum of the cumulative volumes of pockets₆₀₋₅₀₀: which is vol_{pocket}_{₆₀₋₅₀₀} (step E21′).

At the end of this step, arrays of spatially close SL invariant or covariant residues, which are exposed on the surface of the candidate protein and preferably in a pocket having a volume of between 60 Å3 and 500 Å3, will finally be selected. Arrays of at least five target residues are preferred.

In a complementary way, SL invariant or covariant residues can be represented in the form of graphs (step E8). Indeed, from the detected networks, groups of interdependent residues are formed using mathematical graphs. In these graphs, invariant residues and pairs of synthetic lethal residues are integrated to form invariance groups. The nodes of the graph are the residues, the edges define the link between the residues (synthetic lethals or invariants, in which case they are linked to all) and only exist if the two residues are positioned within 10 angstroms of each other and on the surface of the candidate protein. Ideally, this type of graph can be visualized by virtue of the free Graphviz software. These graphs are a means of assessing the quality of the regions identified.

Step E3 consists of determining whether there are any advantageous chemical interactions between the residues and/or between the pairs of residues within the candidate protein, based on the 3D structure of the candidate protein. This step E3 can alternatively be limited to analyzing the advantageous chemical interactions existing between the residues and/or between the pairs of residues selected within the candidate region obtained in step E2, based on the 3D structure of this candidate region.

Genetics enables us to functionally detect the impact of microscopic physico-chemical changes occurring in residues. Thus, the fact of being invariant (or being part of an invariance group) is the consequence of the physico-chemical quality of one or more residues (selection pressure imposes the maintenance of this or these residue(s) in this position). Conversely, if the physico-chemistry of a residue is changed (for example by a mutation or by the external environment), the function and/or the structure of the region may be affected. Based on this observation, it is recommended to take into account the physico-chemical links existing between the residues of the proteins studied, to reinforce or invalidate the results obtained previously, with the aim of identifying the most relevant target regions.

Thus, for each amino acid in the candidate protein, the chemical bonds (for example hydrogen, hydrophobic, ionic and repulsive) wherein this amino acid could potentially participate are determined (step E3 in FIG. 1). In particular, the three-dimensional structure of the protein is used to identify the pairs of residues whose amino acids are sufficiently close so that the chemical bonds identified can effectively influence the function and/or the stability of these residues (see step E4 in FIG. 1).

The additional steps E3 and E4 therefore enable us to identify pairs of amino acids having a chemical interaction that influences their function, or that may change when the environment of the protein changes (pH, temperature, ionic strength, etc.). Indeed protein stability is indeed often pH-dependent, and varies based on the subtype and/or the origin of the host organism. For example, the hemagglutinin (HA) of the human influenza virus is more stable than the hemagglutinin of the avian virus (Galloway S. E. et al.). Furthermore, mutations can lead to changes in the chemical reaction network, and stabilize or destabilize this protein (Byrd-Leotis L. et al.).

The intraprotein physico-chemical network is made up of several types of interactions, such as hydrophobic and hydrogen interactions, as well as salt bridges, negative repulsive and positive repulsive interactions, inter alia (Dyson H. J. et al.; Hubbard R. E. & Kamran Haider M.; Sticke D. F. et al.; Barlow D. J. & Thornton J. M; Harrison J. S. et al.). These interactions ensure that the protein has a very specific shape: that is why they are important. If they were not there, the structure of the protein and certainly its function would be altered. This is why these regions rich in these chemical bonds are important to determine, in the context of the method of the invention.

Unlike hydrophobic and hydrogen interactions, for which pH sensitivity is negligible (or insufficiently documented), electrostatic interactions are strongly affected by pH variations (Harrison, J. S. et al.; Pahari S. et al.). Histidine residues (pKa≈6.4) are biological pH sensors because they are partially charged at neutral pH and positively charged at acidic pH (Pahari S. et al; Kampmann T. et al.). Arginine and lysine (pKa≈13.8 and 10.7) are more basic and invariably protonated under physiological conditions (Pahari S. et al; Fitch, C. A et al.). However, large fluctuations in pKa can occur depending on the microenvironment (Pahari S. et al.; Harris T. K. & Turner G. J.; Harms M. J. et al.; Di Russo N. V. et al.; Baumgart M. et al.). As a result, the pKa of the negatively charged carboxyl group of aspartate and glutamate (pKa≈3.4 and 4.1) approaches the pH values reached during endosomal maturation (Pahari S. et al.; Mellman I. et al.). As a result, the breaking of salt bridges (or at least their weakening if the hydrogen bond remains), can occur if pH<pKa_{carboxylgroup} (Meuzelaar H. et al.). Finally, cation-cation and anion-anion repulsions induce significant destabilization (Harrison J. S. et al.). As pH drops, negative repulsions can also be disrupted and potentially form hydrogen bonds.

The method described herein therefore includes a step of characterizing the chemical interactions existing between all the atoms of the protein, or at least those present in the previously selected candidate region. This step notably aims to identify the following interactions (Hubbard R. E. & Kamran Haider M.; Barlow D. J. & Thornton J. M; Harrison J. S. et al.; Donald J. E. et al.; Freitas R. F. de & Schapira M.; Onofrio A. et al.):
Hydrophobic bonds between non-polar atoms of the type (CB, CG, CE, CD1, CD2, CE2, CE3, CZ2, CZ3, CH2, CE1, CZ, CG1, CG2, CD, CH2) belonging to the following hydrophobic amino acids (ALA, MET, TRP, PHE, TYR, VAL, LEU, ILE, PRO). Atoms having this type of bond will be selected if their distance is less than 4.2 Å.
Hydrogen bonds consisting up of a proton donor atom (OG, OG1, NE2, ND2, ND1, NE2, NZ, NE, NH1, NH2, OH, NE1) belonging to a proton donor amino acid (SER, THR, GLN, ASN, HIS, LYS, ARG, TYR, TRP) and a proton acceptor atom (OG, OG1, OE1, OE2, OD1, OD2, ND1, NE2, OH) belonging to a proton acceptor amino acid (SER, THR, GLU, ASP, GLN, ASN, HIS, TYR). Atoms having this type of bond will be selected when their distance is less than 3.5 Å (Hubbard R. E. & Kamran Haider M.; Sticke D. F. et al.). These hydrogen bonds may consist of interactions between two side chains or between a side chain and the main chain of the protein. The proton donors (N) of the main chain or the proton acceptors of this same chain can belong to any amino acid in the main chain.
There are two types of electrostatic bonds:
Attractive bonds (salt bridges or ionic interaction) when a cation (NE, NH1, NH2, NZ, NE2, ND1) of a positively charged residue (ARG, LYS, HIS), notably when it is at a distance less than 4 Å from an anion (OD1, OD2, OE1, OE2) carried by a negatively charged residue (ASP, GLU).
Repulsive bonds consisting of two atoms with identical charges (−/−) or (+/+), notably when they are separated by a distance of less than 5 Å (Barlow D. J. & Thornton J. M.; Harrison J. S. et al.).

All positively and negatively charged atoms can be taken into account in the calculation, as long as the charges are evenly distributed between the ionizable groups, by stabilizing the resonance between the charges.

Different scores can be calculated to evaluate the chemical interactivity of each amino acid within the candidate region or within the protein. It is notably possible to determine the percentage of hydrophobic bonds and/or the percentage of hydrogen bonds and/or the percentage of salt bridges and/or the percentage of negative repulsions and/or the percentage of positive repulsions and/or the percentage of chemical bonds (step E3′).

In practice, the method of the invention therefore advantageously contains a step of calculating the distance between each atom of the protein, for example from a pdb, SwissProt, uniprot or Modbase file. Preferably, distances between atoms belonging to the same position are not calculated unless they belong to different protomers. Following this calculation, atoms separated by a maximum distance of 5 Å are selected. This step is preferably carried out before recording the chemical interactions between atoms. Thus, only chemical interactions bonding nearby atoms must be taken into account. However, it is possible to perform the two steps in reverse order.

From the number d̅_̅{̅N̅e̅a̅r̅5̅}̅ representing the total number of pairs of atoms within 5 angstroms, several scores can be calculated. It is notably possible to calculate the percentage of hydrophobic bonds and/or the percentage of hydrogen bonds and/or the percentage of salt bridges and/or the percentage of negative repulsions and/or the percentage of positive repulsions and/or the percentage of chemical bonds (step E3′).

It is also possible to calculate a score function ƒ(S_{cochem}) which gives a picture of the overall chemical interactions of the protein studied (step E3′).

By virtue of all these steps, the residues linked by said advantageous chemical interactions, and being at a distance such that these bonds influence the function of these residues, are selected. This is step E4 in FIG. 1.

At this stage, it is possible to represent the various chemical bonds in graph form (step E8′). Indeed, mathematical graphs are formed from the previously detected bonds. The nodes of the graph are the positions/residues, and the edges define the chemical bond between the positions. In a complementary way, this type of graph can be visualized by virtue of the free Graphviz software. These graphs are an additional way of evaluating the bonds identified.

Step E5 identifies the best therapeutic target region(s) for generating a potential drug by cross-referencing the results obtained after step E2 with those obtained after step E4.

The quality of the selected targets can advantageously be evaluated by calculating several score functions. For example, it is possible to calculate the invariance of each target region by the function ƒ(S_{invX}). The invariance of each target region ensures that it will be stable over time. Furthermore, the function ƒ(S_{X}) provides a score between 0 and 1 for each S_{X} target. This score is used to evaluate how effective the identified target is in preserving the drug. In other words, these two score functions can be used to evaluate the effectiveness of the target region.

The target regions can advantageously be represented in the form of graphs (step E8″). Indeed, from the previously detected pairs, groups of interdependent residues are formed by means of mathematical graphs. These graphs integrate the invariant residues and the pairs of synthetic lethal residues that form invariance groups and that are linked by advantageous chemical bonds. The nodes of the graph are the selected positions/residues, the edges define the bond between the positions (synthetic lethal, invariant, or advantageous chemical interaction) and exist only if both residues at that position are positioned within 10 angstroms and on the surface of the candidate protein. In a complementary way, this type of graph can be visualized by virtue of the free Graphviz software. These graphs are an additional means of evaluating the quality of the regions identified.

The complexity of the graphs can be calculated (step E8′″) by score S26 and the number of related graphs by score S27. Such complexity is useful: if the graph is complex, this means there are a lot of targets. If it is very complex, there may be intersections between non-empty targets. If there are many subgraphs, this means that one residue is linked to many others and that the majority of the function falls to it.

### Score Definitions

S1: S_{L}: Sequence length heterogeneity.

Consider μ the mean and σ the standard deviation of the amino acid lengths of the sequences in the sample studied.

$$if σ < μ then , {S}_{L} = 1 - \frac{σ}{μ}$$

$$if σ > μ then {S}_{L} = 0$$

A score S_{L}=1 indicates absolute heterogeneity while S_{L}=0 indicates no heterogeneity.
S2: Sₘᵤₜ_{_{ab}}: Sequences having an aberrant number of mutations.

Consider μ the mean and σ the standard deviation of the number of mutations calculated for each sequence in the sample studied, compared with a reference sequence. The sample studied consists of the polypeptide sequences downloaded before the cleaning steps and the reference sequence introduced in step E0 described above.

Knowing that the number of aberrant mutations is defined as mut_{ab}>=μ+3×σ and that s̅e̅q̅ₘᵤₜ_{_{ab}} corresponds to the number of sequences having a mutation number greater than or equal to mut_{ab}. It is then possible to calculate: Sₘᵤₜ_{_{ab}}=1−(s̅e̅q̅ₘᵤₜ_{_{ab}}÷s̅e̅q̅) with s̅e̅q̅ the total number of sequences.

The closer S2 is to 1, the greater the number of aberrant mutations.
S3: S_{NYP}_{_{ab}}: Sequences having an aberrant number of poorly-defined amino acids (AA) (denoted NYP). In this respect, it is noted that during the sequencing step of the nucleotide sequence, it is sometimes impossible to define exactly which nitrogen base (A, T, G, C) is located at this position although it is possible to affirm that a nucleotide does exist at this position. This position is denoted N. When sequencing cannot define the exact nucleotide, but it is possible to affirm that it is a purine, then a P is added, and if it is a pyrimidine, then a Y is added. In most cases, this makes it impossible to define the amino acid found at this position, which on the protein alignment will be noted as a “gap”. It will therefore no longer be possible to differentiate between a difficulty in determining the nitrogen base during sequencing and the fact that sequencing was not carried out. Thus, from the nucleotide sequences, it is possible to calculate the number of poorly-defined amino acids.

Consider μ the mean and σ the standard deviation of the number of mutations calculated for each sequence in the sample studied, compared with a reference sequence.

Knowing that the number of aberrant NYPs is defined as NYP_{ab}>=μ+3×σ and that s̅e̅q̅_{NYP}_{_{ab}} corresponds to the number of sequences having a number of NYPs greater than or equal to NYP_{ab}. It is then possible to calculate: S_{NYP}_{_{ab}}=1−(s̅e̅q̅_{NYP}_{_{ab}}÷s̅e̅q̅)

The closer S3 is to 1, the greater the number of aberrant amino acids.
S4: S_{gap}_{_{ab}}: Sequences having an aberrant number of gaps.

Consider μ the mean and σ the standard deviation of the number of mutations calculated for each sequence in the sample studied, compared with a reference sequence.

Knowing that the aberrant number of gaps is defined as gap_{ab}>=μ+3 xσ and that s̅e̅q̅_{gap}_{_{ab}} corresponds to the number of sequences having a gap number greater than or equal to gap_{ab}. It is then possible to calculate: S_{gap}_{_{ab}}=1−(s̅e̅q̅_{NYP}_{_{ab}}÷s̅e̅q̅)

The closer score S4 is to 1, the more aberrant the number of gaps.
F(S1QS): Impact of sequence data on statistical prediction quality.

This function varies from 0 to 1. ƒ(S_{1QS}) is used to evaluate whether the input data are sufficient and robust for the prediction to be made:

$$f ( {S}_{1 QS} ) = ( {S}_{{mut}_{a b}} \times 5 + ( {S}_{\overline{\overline{{NYP}_{a b}}}} + {S}_{\overline{\overline{{gap}_{a b}}}} ) \times 3 + {S}_{L} ) \div 9$$

S5: S_{pdb}_{_{ab}}: 3D structure having an aberrant number of missing data.

Let us consider, p̅d̅b̅ as the total number of files studied and μ the mean and a the standard deviation of the number of amino acids per file of the sample studied, compared with a reference file. This reference file can be selected in one of two ways. Either this file is defined as such by the scientific community, or because it represents the root of a phylogenetic tree containing all or most of the proteins studied

Knowing that the number of missing aberrant amino acids (AA) is defined such that:

$$A {A}_{a b} = μ + 3 \times σ$$

And that p̅d̅b̅_{AA}_{_{ab}} is the number of files having a missing AA greater than or equal to mut_{ab}. It is then possible to calculate:

$${S}_{p d {b}_{a b}} = ( 1 - {\overline{\overline{pdb}}}_{A {A}_{a b}} ) \div \overline{\overline{pdb}}$$

It is noted that the choice of a particular reference structure has little impact since its total number of residues will be very close to that of another structure described that does not have an aberrant number of unspecified positions.
S6: S_{subunit} impact of the existence of different subunits on the final result.

If the protein studied has several subunits (each denoted as a subunit), they will be listed, regardless of the number of sequences in the alignment. If it has only one subunit, then this score will be equal to 1.

$${S}_{subunit} = 1 \div \overline{\overline{subunit}}$$

S7: S_{s̅e̅q̅}: Importance of the number of sequences (theoretical threshold at 1000).
s̅e̅q̅ is the total number of sequences in the sample, excluding sequences that have an aberrant number of mutations

$$∀ \overline{\overline{seq}} ∈ ℕ ,$$

$$si ( \overline{\overline{seq}} - ( {\overline{\overline{seq}}}_{{mut}_{a b}} ⋃ {\overline{\overline{seq}}}_{{NYP}_{a b}} ⋃ {\overline{\overline{seq}}}_{g a {p}_{a b}} ) ) > 1000 , {S}_{\overline{\overline{seq}}} = 1$$

$$si ( \overline{\overline{seq}} - ( {\overline{\overline{seq}}}_{{mut}_{a b}} ⋃ {\overline{\overline{seq}}}_{{NYP}_{a b}} ⋃ {\overline{\overline{seq}}}_{g a {p}_{a b}} ) ) < 10 \; 00 , {S}_{\overline{\overline{seq}}} = \overline{\overline{seq}} / 1000$$

S8: S_{gap}_{ₐₗᵢ}: evaluates the impact of gaps in the alignment.

Let us consider μ the mean and σ the standard deviation of the number of gaps calculated per sequence of the sample studied after alignment, compared with the same sequence before alignment.

$$if σ < μ then {S}_{{gap}_{ali}} = 1 - \frac{σ}{μ}$$

$$if σ > μ then {S}_{{gap}_{ali}} = 0$$

S9: Sₑₓₜ_{_{N}} and Sₑₓₜ_{_{C}}: impact of the variance of the 5′ and 3′ ends.

5 amino acids (AA) from the 5′ end and 5 AA from the 3′ end are studied. Invariant residues (inv), pairs of synthetic lethals (SL) and of compensatory mutations (CM) are listed at the ends.

$${S}_{{ext}_{N}} = ( \overline{\overline{{inv}_{N}}} + ( ( \overline{\overline{{SL}_{N}}} - \overline{\overline{{CM}_{N}}} ) \div 2 ) ) \div 5$$

$$unless {S}_{{ext}_{N}} is > 1 in this case {S}_{{ext}_{N}} = 1$$

$${S}_{{ext}_{{3}^{′}}} = ( \overline{\overline{{inv}_{C}}} + ( ( \overline{\overline{{SL}_{C}}} - \overline{\overline{{CM}_{C}}} ) \div 2 ) ) \div 5$$

$$unless {S}_{{ext}_{C}} is > 1 in this case {S}_{{ext}_{C}} = 1$$

S10: S_{red}: Sequence batch redundancy.

Let us consider μ the mean and σ the standard deviation of the histogram of sequence redundancy. The polypeptide sequences corresponding to the candidate protein may be highly heterogeneous or only slightly heterogeneous in terms of the mutations they carry. An extreme situation could be that X sequences all have the same AA sequence. It would then be assumed that this batch is highly homogeneous, that redundancy is absolute, and would therefore have a score equal to 0. Conversely, when the polypeptide sequences are very different, taken in pairs, then there is very little redundancy, and the score tends towards 1.

$$if σ < μ then {S}_{red} = \frac{σ}{μ}$$

$$if σ > μ then {S}_{red} = 1$$

If S10=1 absolute heterogeneity, if S10=0 no heterogeneity
S11: S_{hyp}: impact of hypervariable regions.

The hypervariable regions (hyp) are listed from the bibliography and counted, regardless of the number of sequences in the alignment.

$${S}_{hyp} = 1 \div \overline{\overline{hyp}}$$

S12: S_{indel}: impact of deletions and insertions.

Insertions-deletions (indel) are listed from the bibliography and counted, regardless of the number of sequences in the alignment.

$${S}_{indel} = 1 \div \overline{\overline{indel}}$$

S13: S_{postrad}: impact of post-translational modifications.

The different types of post-translational modifications (postrad) are listed from the bibliography and counted, regardless of the number of sequences in the alignment.

$${S}_{postrad} = 1 \div \overline{\overline{postrad}}$$

S14: S_{subtype}: impact of the existence of different subtypes.

The different subtypes (each denoted “subtype”) belonging to the batch of sequences studied are listed, regardless of the number of sequences in the alignment. Indeed, some microorganisms mutate so rapidly that the evolution over time of these different variants leads to the appearance of variant subtypes.

$${S}_{subtype} = 1 \div \overline{\overline{subtype}}$$

ƒ(S₃): quality of the alignment of primary sequences

$$f ( {S}_{3} ) = ( {S}_{\overline{\overline{seq}}} \times 5 + ( {S}_{{ext}_{N}} + {S}_{{ext}_{C}} ) \div 2 \times 4 + ( {S}_{{gap}_{ali}} + {S}_{indel} ) \div 2 \times 3 + {S}_{L} + {S}_{red} + ( {S}_{hyp} + {S}_{postrad} + {S}_{subtype} ) \div 3 \times 2 ) \div 17$$

ƒ(S_{3QS}): quality of the statistical prediction of the alignment

$$f ( {S}_{3 QS} ) = ( {S}_{L} + ( {S}_{{ext}_{N}} + {S}_{{ext}_{C}} ) \div 2 \times 2 + ( {S}_{{gap}_{ali}} + {S}_{indel} ) \div 2 \times 2 + {S}_{hyp} ) \div 4$$

ƒ(S_{3QC}): impact of the alignment on the target prediction

$$f ( {S}_{3 QC} ) = ( {S}_{\overline{\overline{seq}}} + {S}_{red} + ( {S}_{postrad} + {S}_{subtype} ) \div 2 \times 2 ) \div 3$$

S18: Sₘᵤₜ: Mutational richness of sequences.

Let us consider μ the mean and σ the standard deviation of the number of mutations calculated per sequence in the sample of sequences having a mutation number<mut_{ab}, compared with a reference sequence.

$$if σ < μ then {S}_{mut} = \frac{σ}{μ}$$

$$if σ > μ then {S}_{mut} = 1$$

If S18=1, high heterogeneity, if S18=0 no heterogeneity.
S19: Sᵢₙᵥ: impact of invariant positions.

The percentage of invariant residues (Target Quality QC) is defined. The invariant residues are identified as follows: the same amino acid is found at a given position in at least 99.7% of sequences. The sum of these positions gives i̅n̅v̅

$${S}_{i n v} = \overline{\overline{inv}} \div {L}_{ali}$$

or Lₐₗᵢ is the length of the alignment
ƒ(S_{invariance}): degree of invariance

This score function is used to evaluate the degree of true invariance of the batch of sequences studied, thus giving an image of its essentiality. Indeed, the invariant positions are such that they cannot be selected in the event of mutation since they are essential to the replicability of the organism from which the candidate protein is derived.

$$f ( {S}_{invariance} ) = ( f ( {S}_{3 QC} ) + {S}_{mut} \times 3 + {S}_{inv} \times 2 + {S}_{SL} \times 2 + {S}_{D {′}_{AA}} ) \div 10$$

S20: S_{covar}: impact of the number of covariant positions.

The percentage of covariant residues is defined as follows: sum of the positions found in a pair of covariants (c̅o̅v̅a̅r̅) having a γ² defined according to the Noirvit protocol (Noivirt, et al., 2005).

$${S}_{covar} = \overline{\overline{covar}} \div {L}_{ali}$$

or Lₐₗᵢ is the length of the alignment
S21: S_{prcovar}: impact of the number of pairs of covariant positions.

The percentage of pairs of covariant residues is defined as follows: sum of pairs of residues having a χ² defined according to the Noirvit protocol p̅r̅c̅o̅v̅a̅r̅

$${S}_{prcovar} = \overline{\overline{prcovar}} \div ( {L}_{ali} \times ( {L}_{ali - 1} ) \div 2 )$$

or Lₐₗᵢ is the length of the alignment
S24: S_{D′}_{_{AA}}: impact of overall non-synonymous mutability.

It is calculated as follows:

$${S}_{{D}_{AA}^{′}} = ( {\sum }_{i , j}^{{L}_{ali}} {D}_{AiAj}^{′} ) \div ( {\sum }_{i , j}^{{L}_{ali}} {}^{{′}_{AiAj}} + {\sum }_{i , j}^{{L}_{ali}} {D}_{SiSj}^{′} )$$

D′_{AiAj} and D′_{SiSj} being expressed in Lao et al.
where A denotes a residue of the sequence studied which is non-synonymous with the chosen reference sequence and where S denotes a residue of the sequence studied which is synonymous with the chosen reference sequence.
S25: S_{D′}_{_{SS}}: impact of synonymous mutability.

It is calculated as follows:

$${S}_{{D}_{SS}^{′}} = ( {\sum }_{i , j}^{{L}_{ali}} {D}_{SiSj}^{′} ) \div ( {\sum }_{i , j}^{{L}_{ali}} {D}_{AiAj}^{′} + {\sum }_{i , j}^{{L}_{ali}} {D}_{SiSj}^{′} )$$

D′_{AiAj} and D′_{SiSj} being expressed in Lao et al.
S22: S_{SL}: impact of the number of SLs and their strength on the overall result.

This score is the sum of the negative dissimilarity coefficients ξ therefore of the pairs of SL residues relative to the sum of all the γ² (SL plus CM)

$${S}_{SL} = ( {\sum }_{i , j}^{{L}_{ali}} {\sum }_{A , B}^{AA} {ξ}_{SLAiBj} ) \div ( {\sum }_{i , j}^{{L}_{ali}} {\sum }_{A , B}^{AA} {γ}_{AiBj}^{2} )$$

S23: S_{CM}: impact of the number and strength of CMs on the overall result.

This score is the sum of the positive dissimilarity coefficients ξ therefore of the pairs of CM residues relative to the sum of all the γ² (SL plus CM)

$${S}_{CM} = ( {\sum }_{i , j}^{{L}_{ali}} {\sum }_{A , B}^{AA} {ξ}_{CMAiBj} ) \div ( {\sum }_{i , j}^{{L}_{ali}} {\sum }_{A , B}^{AA} {γ}_{AiBj}^{2} )$$

S27: S_{MEdge}: Complexity of the graph.

A graph G is defined by a pair (S,A) with S a finite set of vertices, and A a finite set of pairs of vertices (sᵢ, sⱼ) in S². A pair is therefore a pair of vertices linked by an edge

The aim is to study the invariant and SL residues involved in several pairwise relationships (close in space and located on the protein surface, being either invariant or involved in a synthetic lethality relationship). From the graph of bonds, this score is defined as being the mean number of edges (a) per node (ι̅)

$${S}_{MEdge} = ( {\sum }_{i} a ) \div \overline{\overline{ι}}$$

S28: S_{connect}: Number of related graphs. A graph is related if each pair of vertices is connected by an edge.
S28: S_{Near5}: residues close in space (5 angstroms).

This score corresponds to the proportion of pairs of residues located within 5 angstroms of each other (Cα). Starting from a reference pdb file, the distance between the 2 Cα of a pair of residues is calculated for all pairs of residues. The number of pairs separated by less than 5 angstroms is evaluated and denoted d̅_̅{̅N̅e̅a̅r̅5̅}̅.

$${S}_{Near 5} = \overline{\overline{{d}_{Near 5}}} \div ( {L}_{ali} \times ( {L}_{ali - 1} ) \div 2 )$$

S29: S_{Near10}: residues close in space (10 angstroms).

This score corresponds to the proportion of pairs of residues located within 10 angstroms of each other (Cα). Starting from a reference pdb file, the distance between the 2 Cα of a pair of residues is calculated for all pairs of residues. The number of pairs separated by less than 10 angstroms is denoted d̅_̅{̅N̅e̅a̅r̅1̅0̅}̅.

$${S}_{Near 10} = \overline{\overline{{d}_{Near 10}}} \div ( {L}_{ali} \times ( {L}_{ali - 1} ) \div 2 )$$

S30: S_{NearinvSL5}: invariants and SL close in space (5 angstroms)

Starting from a reference pdb file, the distance between the 2 Cα of a pair of residues is calculated for all pairs of residues. The number of pairs separated by less than 5 angstroms is evaluated and denoted d̅_̅{̅N̅e̅a̅r̅I̅n̅v̅S̅L̅5̅}̅.

$${S}_{NearInvSL 5} = \overline{\overline{{d}_{NearInvSL 5}}} \div ( {L}_{ali} \times ( {L}_{ali - 1} ) \div 2 )$$

S31: S_{NearInvSL10} invariant and SL close in space (10 angstroms)

Starting from a reference pdb file, the distance between the 2 Cα of a pair of residues is calculated for all pairs of residues. The number of pairs separated by less than 10 angstroms is denoted d̅_̅{̅N̅e̅a̅r̅I̅n̅v̅S̅L̅1̅0̅}̅.

$${S}_{NearInvSL 10} = \overline{\overline{{d}_{NearInvSL 10}}} \div ( {L}_{ali} \times ( {L}_{ali - 1} ) \div 2 )$$

S32: S_{acc}: Percentage of accessible residues.

$${S}_{acc} = \overline{\overline{acc}} \div ( \overline{\overline{acc}} + \overline{\overline{enf}} )$$

Or a̅c̅c̅ represents the number of accessible residues and b̅u̅r̅ the number of buried residues, can be determined by ASA software (Alland et al., 2005)
S33: S_{acc}_{_{invSL}}: Percentage of accessible invariant and SL residues.

$${S}_{{acc}_{InvSL}} = \overline{\overline{{acc}_{InvSL}}} \div ( \overline{\overline{{acc}_{InvSL}}} + \overline{\overline{{bur}_{InvSL}}} )$$

Or a̅c̅c̅_̅{̅I̅n̅v̅S̅L̅}̅ represents the number of accessible residues and b̅u̅r̅_̅{̅I̅n̅v̅S̅L̅}̅ the number of buried residues. They are determined by ASA software (Alland et al., 2005)

$$f ( {S}_{cochem} ) \text{:} chemical reactivity \text{:} f ( {S}_{cochem} ) = ( {S}_{cochemTot} \times 5 + {S}_{{pdb}_{ab}} + {S}_{\overline{\overline{pdb}}} \times 2 ) \div 8$$

With S_{cochemTot} the percentage of chemical bonds, S_{p̅d̅b̅}
ƒ(S_{invX}): Invariance score for each target

Two sets A and B are defined:
Set A groups together related or very loosely related graphs (each of these sub-graphs represents a subset of set A). Let us recall the definition of an edge linking two residues: the two residues are less than 10 angstroms apart, they are present on the surface of the molecule and are either invariant or form part of an SL pair.
Set B groups together the targets defined by the Fpocket software (each target defines a subset of set B).

The next step is to link a subset of set A to a subset of set B. Once these subsets have been linked in pairs (one from set A to one from set B), the intersection of their elements (interₓ) and their union is defined (unionₓ).

$$f ( {S}_{invX} ) = ( \overline{\overline{{inter}_{x}}} ) \div ( \overline{\overline{{union}_{x}}} )$$

The closer (i̅n̅t̅e̅r̅ₓ̅)÷(u̅n̅i̅o̅n̅ₓ̅) is to 1, the more invariant the target x is, and therefore the less subject to variability it will be in the future. Such a target will thus drastically reduce the emergence of new drug-resistant variants. Indeed, for these variants to exist, positions on the target would have to be mutated. However, such a mutated target would no longer allow the variant to be replicated and would therefore call into question its existence.
ƒ(S_{X}): score between 0 and 1 for each target

$$f ( {S}_{X} ) = ( {vol}_{x} \div {vol}_{{pocket}_{60 - 500}} + f ( {S}_{invX} ) + {S}_{chemTot} \div \overline{\overline{{inter}_{x}}} + ( {S}_{cochemTot} \div ( {inter}_{x} \times ( {inter}_{x} - 1 ) ) \div 2 ) ) ) \div 4$$

This scoring function assigns a score to each of the targets determined by our software and defines the “druggability” of this target. “Druggability” means being able to efficiently bind a small molecule with therapeutic potential, therefore representing a future drug. It is added to this definition that this target would leave little or no possibility of therapeutic escape. Thus, a small molecule, a future drug, must be able to chemically bind to a group of residues (S_{cochemTot}) located in a concave space (called a pocket), whose volume (volₓ) can be determined, and having the greatest possible invariance (ƒ(S_{invX}).

$$f ( {S}_{4} ) \text{:} three - dimensional alignment quality \text{:} f ( {S}_{2} ) = ( {S}_{{pdb}_{ab}} \times 8 + {S}_{\overline{\overline{pdb}}} \times 5 + {S}_{TM - pdb} \times 3 + {S}_{res} \times 3 ) \div 19$$

$$f ( {S}_{4 QS} ) \text{:} quality of statistical prediction of the structural alignment \text{:} f ( {S}_{2 QS} ) = ( {S}_{{pdb}_{ab}} \times 8 + {S}_{\overline{\overline{pdb}}} \times 5 + {S}_{res} \times 3 ) \div 16$$

$$f ( {S}_{4 QC} ) \text{:} Impact of three - dimensional alignment on target prediction \text{:} f ( {S}_{2 QC} ) = ( {S}_{\overline{\overline{pdb}}} \times 5 + {S}_{TM - pdb} \times 3 ) \div 8$$

### Example

The method of the invention has been implemented on the influenza virus hemagglutinin (HA) protein.

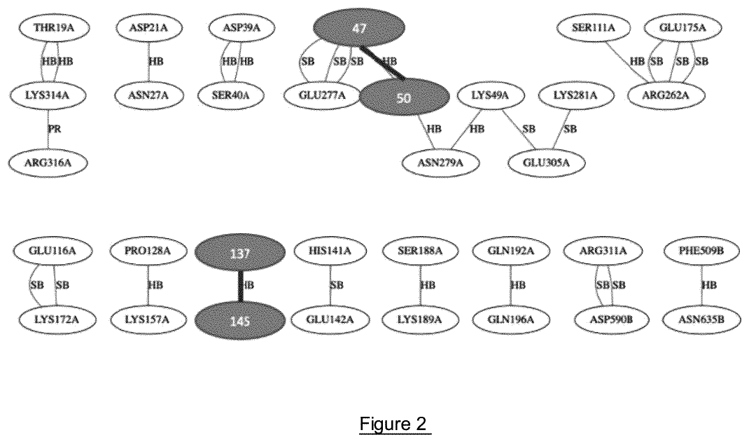

This implementation highlighted several advantageous chemical bonds enabling the selection of 7 amino acid pairs/groups linked by important chemical and genetic bonds, with acceptable distances therebetween (FIG. 2, see the shaded amino acids).

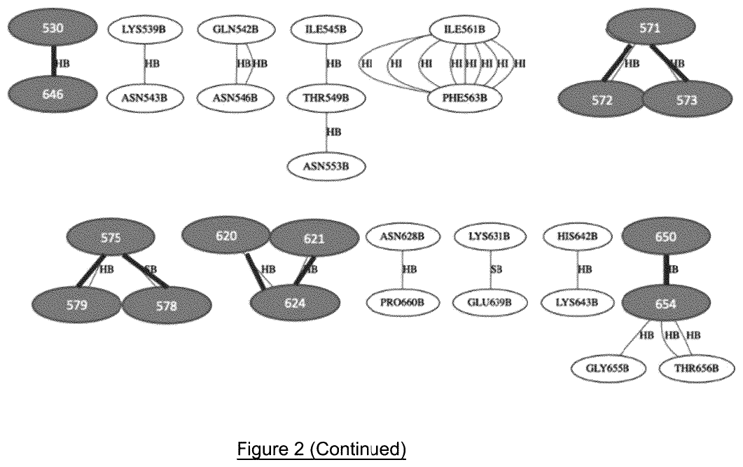

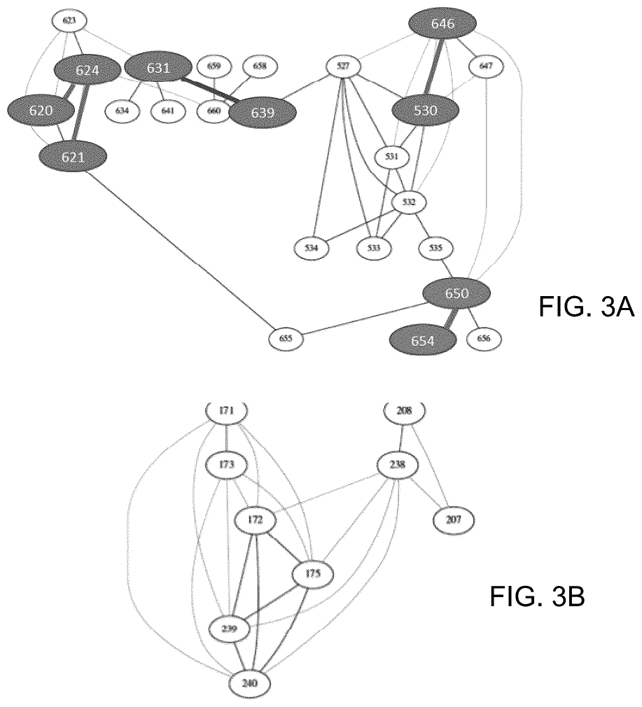

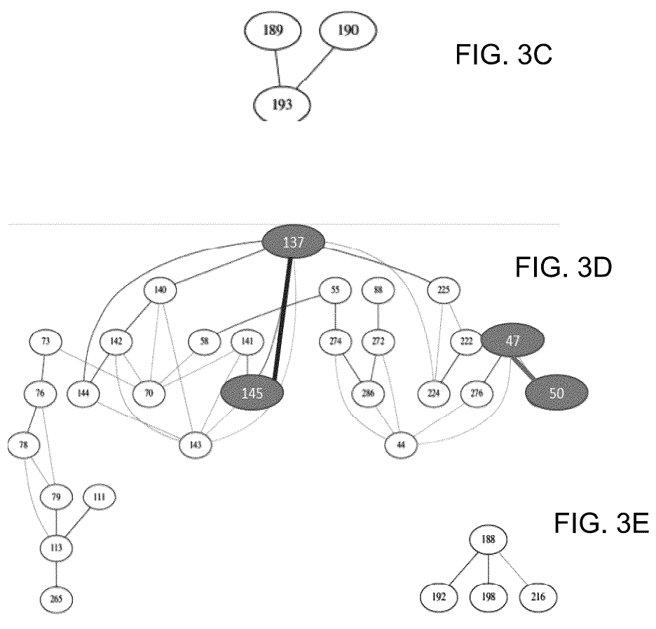

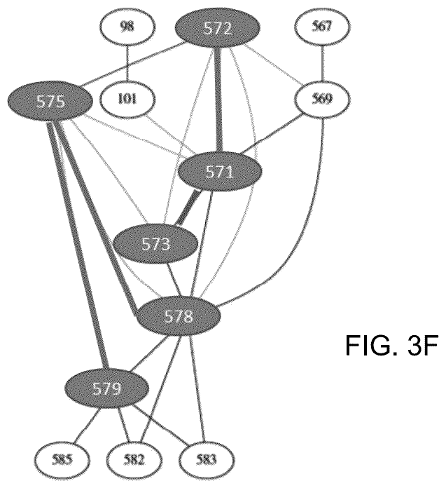

These particularly advantageous pairs/groups were then taken into account in the analysis of the therapeutic target regions of the HA protein identified during the implementation of steps (E1) and (E2) of the method of the invention. As shown in FIG. 3 (using information from FIG. 3 of Lao et al.), the inclusion of these 7 preferential amino acid groups reduces the number of therapeutic target regions within HA from 6 to 3. These 3 therapeutic targets will therefore be the focus of pharmaceutical drug screening with an increased likelihood of identifying reliable and effective active ingredients.

The method of the invention therefore refines the method previously proposed in Lao et al. by adding a step (E3) which requires the determination of all chemical interactions present between each residue and/or between each pair of residues within the candidate protein separated by a distance of at most 5 angstroms (see FIG. 2). By virtue of this additional step, only those pairs/groups of residues having advantageous physico-chemical characteristics and located at such a distance that the chemical links between residues potentially have an influence (E4), will be selected.

Thus, the “chemical interactivity” information proposed in the present invention reinforces the validity of the targets identified by the genetic approach of Lao et al. by selecting the most relevant, so as to significantly reduce the number of therapeutic targets to be used in molecule library screening programs and to enhance the likelihood of identifying more effective drugs faster.

### BIBLIOGRAPHIC REFERENCES

Alland et al., 2005 Alland et al.: Alland C, Moreews F, Boens D, Carpentier M, Chiusa S, Lonquety M, Renault N, Wong Y, Cantalloube H, Chomilier J, et al. 2005. RPBS: a web resource for structural bioinformatics. Nucleic Acids Res 33:W44-49
Barlow, D. J. & Thornton, J. M. Ion-pairs in proteins. Journal of Molecular Biology 168, 867-885 (1983)
Baumgart, M. et al. Design of buried charged networks in artificial proteins. Nat Commun 12, 1895 (2021).
Brouillet S., Valere T., Ollivier E., Marsan L., Vanet A., Co-lethality studied as an asset against viral drug escape: the HIV protease case. Biol Direct. 2010 Jun. 17; 5:40.
Byrd-Leotis, L., Galloway, S. E., Agbogu, E. & Steinhauer, D. A. Influenza hemagglutinin (HA) stem region mutations that stabilize or destabilize the structure of multiple HA subtypes. J Virol 89, 4504-4516 (2015).
Cheng C, Xiao P. Evaluation of the correctable decoding sequencing as a new powerful strategy for DNA sequencing. Life Sci Alliance. 2022 Apr. 14; 5(8):e202101294.
Childers, M. C., Towse, C.-L. & Daggett, V. The effect of chirality and steric hindrance on intrinsic backbone conformational propensities: tools for protein design. Protein Eng Des Sel 29, 271-280 (2016).
Di Russo, N. V., Estrin, D. A., Marti, M. A. & Roitberg, A. E. pH-Dependent conformational changes in proteins and their effect on experimental pK(a)s: the case of Nitrophorin 4. PLoS Comput Biol 8, el002761 (2012).
Donald, J. E., Kulp, D. W. & DeGrado, W. F. Salt bridges: geometrically specific, designable interactions. Proteins 79, 898-915 (2011).
Dyson, H. J., Wright, P. E. & Scheraga, H. A. The role of hydrophobic interactions in initiation and propagation of protein folding. Proceedings of the National Academy of Sciences 103, 13057-13061 (2006).
Fitch, C. A., Platzer, G., Okon, M., Garcia-Moreno E, B. & McIntosh, L. P. Arginine: Its pKa value revisited. Protein Sci24, 752-761 (2015).
Freitas, R. F. de & Schapira, M. A systematic analysis of atomic protein-ligand interactions in the PDB. Med. Chem. Commun. 8, 1970-1981 (2017).
Galloway, S. E., Reed, M. L., Russell, C. J. & Steinhauer, D. A. Influenza HA subtypes demonstrate divergent phenotypes for cleavage activation and pH of fusion: implications for host range and adaptation. PLoS Pathog 9, e1003151 (2013).
Harms, M. J. et al. The pKa Values of Acidic and Basic Residues Buried at the Same Internal Location in a Protein Are Governed by Different Factors. Journal of Molecular Biology 389, 34-47 (2009).
Harris, T. K. & Turner, G. J. Structural Basis of Perturbed pKa Values of Catalytic Groups in Enzyme Active Sites. IUBMB Life 53, 85-98 (2002).
Harrison, J. S. et al. Role of Electrostatic Repulsion in Controlling pH-Dependent Conformational Changes of Viral Fusion Proteins. Structure 21, 1085-1096 (2013).
Hubbard, R. E. & Kamran Haider, M. Hydrogen Bonds in Proteins: Role and Strength in ELS (John Wiley & Sons, Ltd, 2010).
Kampmann, T., Mueller, D. S., Mark, A. E., Young, P. R. & Kobe, B. The Role of Histidine Residues in Low-pH-Mediated Viral Membrane Fusion. Structure 14, 1481-1487 (2006).
Kuiken H. J., Beijersbergen R. L., Exploration of synthetic lethal interactions as cancer drug targets. Future Oncol. 2010 November; 6(11):1789-802
Lao J. and Vanet A. A New Strategy to Reduce Influenza Escape: Detecting Therapeutic Targets Constituted of Invariance Groups. Viruses. 2017 Mar. 2; 9(3):38.
Mellman, I., Fuchs, R. & Helenius, A. Acidification of the endocytic and exocytic pathways. Annu. Rev. Biochem. 55, 663-700 (1986).
Meuzelaar, H., Vreede, J. & Woutersen, S. Influence of Glu/Arg, Asp/Arg, and Glu/Lys Salt Bridges on α-Helical Stability and Folding Kinetics. Biophysical Journal 110, 2328-2341 (2016).
Le Guilloux, V.; Schmidtke, P.; Tuffery, P. Fpocket: An open source platform for ligand pocket detection. BMC Bioinform. 2009, 10, 168
Noivirt, O.; Eisenstein, M.; Horovitz, A. Detection and reduction of evolutionary noise in correlated mutation analysis. Protein Eng. Des. Sel. 2005, 18, 247-253
Onofrio, A. et al. Distance-dependent hydrophobic-hydrophobic contacts in protein folding simulations. Phys. Chem. Chem. Phys. 16, 18907-18917 (2014).
Pahari, S., Sun, L. & Alexov, E. PKAD: a database of experimentally measured pKa values of ionizable groups in proteins. Database 2019, baz024 (2019).
Petitjean M., Badel A., Veitia R. A., Vanet A., Synthetic lethals in HIV: ways to avoid drug resistance: Running title: Preventing HIV resistance. Biol Direct. 2015 Apr. 17; 10:17
Sticke, D. F., Presta, L. G., Dill, K. A. & Rose, G. D. Hydrogen bonding in globular proteins. Journal of Molecular Biology 226, 1143-1159 (1992).

1. A computer-implemented method for determining at least one therapeutic target region on a candidate protein of a target pathogenic organism, said method comprising the following steps:
a) Identifying (E1), in a set of previously aligned nucleotide and polypeptide sequences characteristic of said candidate protein, invariant residues and/or pairs of synthetic lethal residues referred to as target residues;
b) Identifying (E2) at least one candidate region consisting of at least one pair of target residues identified in step a), said at least one pair comprising target residues located at a determined distance in space and being exposed at the surface of the candidate protein, preferably in a pocket;
c) Determining (E3) the advantageous chemical interactions between each residue and/or between each pair of residues within the candidate protein, from the 2D and/or 3D structure of the candidate protein; said advantageous chemical interactions being hydrophobic bonds and/or hydrogen bonds and/or salt bridges and/or negative-repulsion and/or positive-repulsion bonds;
d) Selecting (E4) the residues linked by said advantageous chemical interactions, said residues being at a distance of at most 10 angstroms;
e) Selecting (E5) at least one therapeutic target region from among the candidate regions identified in step b), comprising the residues selected in step d).

2. The method according to claim 1, wherein the step (E3) of identifying advantageous chemical interactions consists of determining the chemical interactivity of all residues within the protein and/or determining the chemical interactivity of all residue pairs within the protein, said residues or residue pairs being separated by a determined distance of at most 10 angstroms.

3. The method according to claim 1, wherein the distance between the invariant residues or between the synthetic lethal pairs determined in step b) is between 2 and 8 angstroms, preferably 5 angstroms.

4. The method according to claim 1, comprising an evaluation (E3′) of the chemical interactivity of the bonds determined (E3) comprising a calculation of at least one score from: a percentage of hydrophobic bonds and/or a percentage of hydrogen bonds and/or, a percentage of salt bridges and/or a percentage of negative repulsion bonds and/or a percentage of positive repulsion bonds and/or a percentage of chemical bonds.

5. The method according to claim 1, wherein the target region is a pocket identified from a set of aligned 3D structures of the candidate protein and wherein the chemical interactions are determined on a set of aligned 3D structures of the candidate protein.

6. The method according to claim 5, wherein the target pocket comprises at least five target residues selected from synthetic lethal invariant residues or covariant residues, and wherein said pocket has a volume between 60 \dot{A}³ and 500 \dot{A}³.

7. The method according to claim 1 comprising an evaluation of the quality of the spatial location of target residues, said evaluation being characterized by calculating the proportion of residue pairs located within 5 angstroms of each other and/or the proportion of residue pairs located within 10 angstroms of each other.

8. The method according to claim 1, comprising a step (E0) of selecting the reference polypeptide sequence of the candidate protein, followed by a step of selecting the polypeptide test sequences having lengths identical to the reference sequence, and/or having a number of mutations less than or equal to three standard deviations of the mean number of mutations per sequence, and/or having an invariant residue at its N or C end.

9. The method according to claim 8, comprising an evaluation (E11′) of the quality of the selected polypeptide sequences, said evaluation comprising calculating at least one score measuring the heterogeneity of the length of the selected polypeptide sequences and/or the number of selected polypeptide sequences having an aberrant number of poorly-defined amino acids and/or the number of sequences having an aberrant number of missing residues compared to the reference polypeptide sequence of the candidate protein.

10. A computer-implemented method for determining at least one therapeutic target region on a candidate protein of a target pathogenic organism, said method comprising the following steps:
a) Determining (E3) the advantageous chemical interactions between each residue and/or between each pair of residues within the candidate protein, from the 2D and/or 3D structure of the candidate protein; said advantageous chemical interactions being hydrophobic bonds and/or hydrogen bonds and/or salt bridges and/or negative-repulsion and/or positive-repulsion bonds; and
b) Selecting the residues (E4) linked by said advantageous chemical interactions, said residues being at a distance of at most 10 angstroms, said residues forming a therapeutic target region.
